(19) United States
(12) Patent Application Publication
(10) Pub. No.: US 2024/0358248 A1
(43) Pub. Date: Oct. 31, 2024

(54) ENDOSCOPE REPROCESSING SYSTEM AND TECHNIQUE TO DETECT DISCONNECTION OF AN ENDOSCOPE BIOPSY CHANNEL

(71) Applicant: Ecolab USA Inc., Saint Paul, MN (US)

(72) Inventors: Laurent BERENGUER, Aubagne (FR); Christian AFFRE, Allauch (FR); Eric PENDARIES, La Ciotat (FR)

(21) Appl. No.: 18/645,816

(22) Filed: Apr. 25, 2024

Related U.S. Application Data
(60) Provisional application No. 63/499,097, filed on Apr. 28, 2023.

(51) Int. Cl. A61B 1/12; A61B 10/04; G01P 13/00

(52) U.S. Cl. CPC A61B 1/125; A61B 10/04; G01P 13/0006

(57) ABSTRACT

A method of detecting connectivity of a biopsy channel of an endoscope during an endoscope reprocessing procedure may involve connecting a fluid supply line to the biopsy channel and a fluid detection sensor to a suction connector on the endoscope. After connection, a fluid can be introduced into the fluid supply line connected to the biopsy channel of the endoscope. The reprocessing machine can determine whether the fluid supply line is adequately connected to the biopsy channel of the endoscope based on detection of the fluid by the fluid detection sensor.

### RELATED APPLICATION

This application claims priority to U.S. Provisional Application No. 63/499,097, filed Apr. 28, 2023, the entire contents of which are herein incorporated by reference.

### TECHNICAL FIELD

This disclosure relates to systems and techniques for reprocessing reusable medical equipment and, more particularly, systems and techniques for reprocessing endoscopes.

### BACKGROUND

An endoscope is a slender, tubular optical instrument used as a viewing system for examining an inner part of the body and, when used with an attached instrument, for biopsy or surgery. Decontamination systems can be used to reprocess previously-used medical devices, such as endoscopes such that the devices can be used again on a subsequent patient. During the decontamination process of an endoscope, the endoscope can be inserted into a chamber of a reprocessing machine and the internal channels of the endoscope connected to the machine to receive cleaning and/or disinfecting agents. For example, the reprocessing machine may provide a system of lines, pumps and valves for the purpose of feeding a cleaning and/or disinfecting agent to the endoscope placed in a chamber.

To ensure that the internal channels or lumens of the endoscope are adequately cleaned, the individual channels of the endoscope may be fluidly connected to the reprocessing machine by one or more releasable connectors. If a connector is not appropriately attached or otherwise fails to achieve a fluid-tight seal, the lumens of the endoscope may not receive the cleaning and/or disinfecting medium necessary to ensure that the inner surfaces of the lumen have been adequately cleaned and/or disinfected.

Accordingly, configuring a reprocessing machine to assess the adequacy of one or more fluid connections between the machine and endoscope can be beneficial to detect potential problems and help ensure adequate reprocessing of the endoscope.

### SUMMARY

In general, this disclosure is directed to devices, systems, and techniques for reprocessing medical instruments and, more particularly, endoscopes. In particular, the disclosure describes devices, systems, and techniques for determining the adequacy of a connection between an endoscope reprocessing machine and the biopsy channel of an endoscope in order to determine when the biopsy channel is adequately connected to proceed with a reprocessing operation.

While specific endoscope styles and configurations can vary, endoscopes are generally configured as flexible tubular elongated bodies that are insertable into an internal cavity of a patient. The endoscope is divided into multiple discrete lumens or channels, such as one or more channels through which gas (e.g., air) and/or fluid (e.g., water) is delivered and/or suction is drawn. The endoscope can also have a biopsy channel through which medical accessories can be passed, e.g., to biopsy tissue in the body of the patient. The biopsy channel typically has a significantly larger diameter than the diameter of other channels of the endoscope, such as two to ten larger. The biopsy channel can extend from a biopsy port at a control section of the endoscope through to a biopsy channel outlet at the end of the endoscope.

During reprocessing, the individual channels of the endoscope can be fluidly connected to an endoscope reprocessing machine, referred to as an automated endoscope reprocessor (“AER”). The endoscope reprocessing machine can deliver one or more fluids (e.g., water, cleaning liquid, disinfecting liquid, air) to the individual lumens of the endoscope during reprocessing. After connecting the endoscope to the reprocessing machine, the reprocessing machine may perform an operation to check the integrity of the connections between the machine and the endoscope before proceeding with a cleaning operation. For example, the reprocessing machine may control introduction of one or more fluids into fluid pathways intended to be fluidly coupled to corresponding lumens of the endoscope. The reprocessing machine can detect and/or measure the fluid discharging from an opposed end of the lumens and determine therefrom whether the lumen is adequately connected to the reprocessing machine or whether a connection error appears to exist (e.g., in which case fluid does not flow through the lumen and/or the amount of fluid flowing through the lumen does not correspond to the amount introduced into the lumen).

In practice, it can be difficult to determine whether the biopsy channel of an endoscope is adequately connected to the reprocessing machine because of the large size of the biopsy channel. Depending on the configuration of the endoscope reprocessing machine, the machine may typically monitor the flow rate of fluid discharging from the individual lumens of the endoscope to evaluate the connection between the reprocessing machine and the endoscope. However, because the biopsy channel is so comparatively large, fluid introduced into the biopsy channel can flow very rapidly through the channel (e.g., discharging from the outlet end in less than a second), making it difficult to accuracy detect when the biopsy channel is properly connected.

In accordance with some implementations of the present disclosure, automated endoscope reprocessing machines and associated techniques are described that determine whether the biopsy channel of an endoscope is suitably connected to the reprocessing machine by detecting fluid through a different outlet of the endoscope that is fluidly connected to the biopsy channel. In particular, in some examples, automated endoscope reprocessing machines and associated techniques are described that involve connecting a suction channel of an endoscope to a fluid detection sensor associated with the machine. The biopsy channel of the endoscope can be connected to the automated endoscope reprocessing machine and a fluid introduced into the biopsy channel to test whether the biopsy channel is suitably connected to the machine. Example fluids include gases (e.g., air) and liquids (e.g., water). Instead of attempting to monitor a corresponding amount of fluid discharging from the outlet end of the biopsy channel, the fluid detection sensor can be positioned and configured to detect fluid at an inlet of the suction channel of the endoscope, which is fluidly connected to the biopsy channel inside of the endoscope. If the biopsy channel is not suitably connected to the reprocessing machine, fluid introduced into the biopsy channel may not flow back to the inlet of the suction channel. However, if the biopsy channel is suitably connected, fluid introduced into the biopsy channel may flow back to the inlet of the suction channel and be detected by the fluid detection sensor. In this way, the reprocessing machine can evaluate whether the biopsy channel of the endoscope is properly connected before proceeding with subsequent steps of the cleaning process.

In one example, a method of detecting connectivity of a biopsy channel of an endoscope during an endoscope cleaning procedure is described. The method includes connecting a fluid supply line of an endoscope reprocessing machine to a biopsy channel of an endoscope and connecting a fluid detection sensor of the endoscope reprocessing machine to a suction connector of the endoscope. The method further involves introducing a fluid into the fluid supply line connected to the biopsy channel of the endoscope and either (a) detecting, by the fluid detection sensor, the fluid introduced into the fluid supply line connected to the biopsy channel via discharge from the suction connector of the endoscope or (b) failing to detect, by the fluid detection sensor, the fluid introduced into the fluid supply line connected to the biopsy channel via discharge from the suction connector of the endoscope.

In another example, an endoscope reprocessing machine is described that includes a processing chamber configured to receive an endoscope to be reprocessed, a fluid supply line configured to connect to a biopsy channel of the endoscope, and a fluid detection sensor configured to connect to a suction connector of the endoscope. The machine also includes a controller configured to control introduction of a fluid into the fluid supply line connected to the biopsy channel of the endoscope and determine if the fluid supply line is adequately connected to the biopsy channel of the endoscope based on detection of the fluid by the fluid detection sensor.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a block diagram of one example configuration of an AER that can detect connectivity of an endoscope biopsy channel to the machine.

FIG. 2 is a schematic cross-section illustration of an example configuration of an endoscope.

FIG. 3 is a schematic diagram showing an example configuration of the AER of FIG. 1.

FIG. 4 is a flow diagram illustrating an example technique for detecting connectivity of a biopsy channel of an endoscope during an endoscope reprocessing procedure.

### DETAILED DESCRIPTION

This disclosure generally relates to endoscope reprocessing machines and related endoscope reprocessing techniques for processing an endoscope previously inserted into and used on a patient to be suitable for reuse on a subsequent patient. In some examples, the disclosed systems and techniques are implemented to evaluate the quality of a connection made between the endoscope reprocessing machine and the biopsy channel of an endoscope being reprocessed in the machine. If the connection between the biopsy channel of the endoscope and the reprocessing machine is not suitably fluid tight, fluid delivered by the machine through the outlet of the connection may not pass through the biopsy channel but may instead leak at the connection. If this occurs, the biopsy channel may not be suitably processed within the endoscope reprocessing machine.

In some implementations, the quality of the connection between the biopsy channel of the endoscope and the endoscope reprocessing machine is evaluated by fluidly connecting the biopsy channel to the machine, delivering a fluid into the biopsy channel, and monitoring the presence or absence of that fluid delivered into the biopsy channel at the inlet of a different channel of the endoscope. For example, a test fluid may be introduced by the endoscope reprocessing machine at the inlet of the biopsy channel of the endoscope and a fluid detection sensor fluidly connected to the inlet of the suction channel of the endoscope. If the fluid detection sensor detects fluid at the inlet of the suction channel in response to the fluid being introduced into the biopsy channel, the endoscope reprocessing machine may determine that the biopsy channel of the endoscope is suitably connected to the machine and proceed with automated reprocessing steps. By contrast, if the endoscope reprocessing machine does not detect fluid at the inlet of the suction channel in response to the fluid being introduced into the biopsy channel (or, in other implementations, detect a suitable volume and/or flow rate of fluid), the endoscope reprocessing machine may determine that the biopsy channel of the endoscope is not suitably connected to the machine. If this occurs, the endoscope reprocessing machine may issue one or more user alerts and/or prevent further processing of the endoscope (e.g., prevent performing cleaning and/or sterilization steps) until the connection error is rectified.

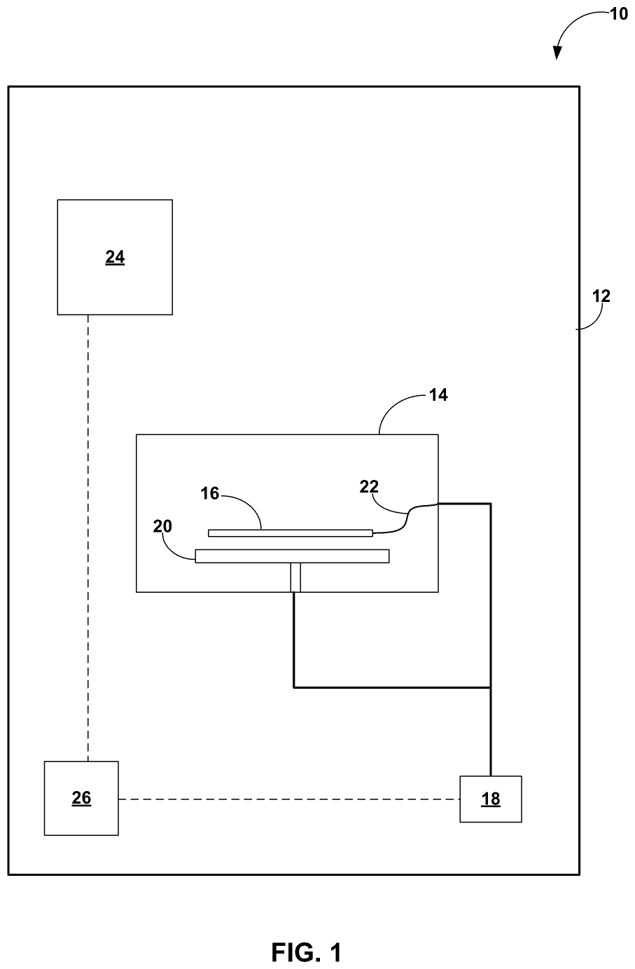

An automated endoscope reprocessing machine (AER) implementing the concepts and techniques of the disclosure can have a variety of different features and configurations. FIG. 1 is a block diagram of one example configuration of an AER that can detect connectivity of an endoscope biopsy channel to the machine according to the disclosure. In the example of FIG. 1, an AER 10 is shown in that includes an exterior housing 12 arranged to contain and provide protection for the components of the AER. AER 10 includes one or more processing chambers 14 configured to receive a medical device 16 to be processed therein, such as an endoscope 16. AER 10 also includes one or more doors to provide access to each processing chamber 14 for loading endoscope 16 in the chamber, sealing the chamber during processing, and for removing the endoscope from the processing chamber after processing. In different implementations, the AER 10 can be configured as a basin-style machine in which endoscope 16 is loaded into and unloaded from processing chamber 14 through a top or upper opening or as a pass-through style machine in which endoscope 16 is loaded through a first door one side of the machine into processing chamber 14 (e.g., a dirty or non-sterile side) and unloaded from the processing chamber through a second door on an opposite side of the machine (e.g., a clean or sterile side).

In use, an operator may place endoscope 16 into an endoscope carrier that is then positioned in processing chamber 14. The endoscope carrier may be a basket have a wire frame or lattice structure that allows ingress and egress of fluids into and out of the carrier once positioned inside of processing chamber 14. In some examples, the operator can fluidly connect each of the channels of endoscope 16 to a connector on the endoscope carrier. The connector carried by the endoscope carrier can then be mechanically and/or fluidly connected to a corresponding connector within processing chamber 14 to fluidly couple the individual channels to AER 10. This can simplify the process of connecting endoscope 16 to AER 10 rather than inserting the endoscope into processing chamber 14 and then connecting each individual channel of the endoscope to the machine within the processing chamber.

AER 10 can include a circulation system that can circulate one or more reprocessing fluids 18 such as detergent, sterilant, disinfectant, water, alcohol, air, and/or any other suitable fluid, for example, through endoscope 16, partially or fully immerse the endoscope in the fluid within processing chamber 14, and/or spray the fluid onto the exterior surface of the endoscope. The circulation system can include a fluid supply and a circulation pump, where the circulation pump can be fluidly connected to the fluid supply such that the fluid can be drawn from the fluid supply into the circulation system. In certain implementations, the circulation system can include a mixing chamber in which the fluid can be mixed with another fluid, such as water, for example, to form a mixed or dilute fluid for delivery to processing chamber 14 and endoscope 16 therein.

In either event, processing chamber 14 can include one or more spray nozzles 20 which can be in fluid communication with the one or more fluid 18, e.g., via a circulation pump such that the fluid pressurized by the circulation pump can be ejected from the circulation system through the spray nozzles and onto an exterior surface of endoscope 16. In some examples, each processing chamber 14 includes a plurality of spray nozzles 20 positioned around the perimeter thereof and/or one or more spray nozzles which can spray upwardly from the floor of the processing chamber.

For example, in some implementations, processing chamber 14 includes one or more rotating arm members. The rotating arm members can be rotatably mounted via a central hub sleeve rotatably connected around a rotating arm hub. Each spray arm can define a spray arm lumen. The spray arm lumens can extend at least a portion of the length of the spray arm and serve to operatively connect a hub sleeve lumen defined within the central hub sleeve with a plurality of spray jets. Together the interconnected hub sleeve lumen, spray arm lumens, and outlet spray openings can provide a conduit for discharging pressurized fluids.

In order to clean, disinfect, and/or sterilize internal channels within endoscope 16, AER 10 can include one or more supply lines 22 in fluid communication with the one or more fluids 18 (e.g., via a circulation system pump) that can be placed in fluid communication with the internal channels and/or ports of the endoscope. In some examples, processing chamber 16 can include one or more complementary connectors that comprise the ends of the supply lines. The individual channels and/or ports of endoscope 16 can be fluidly connected to a master connector which, in term, is connected to the complementary connector inside of AER 10. In some examples, AER 10 can further include one or more flexible conduits which can be connected and/or sealingly engaged with the ports and/or the channels defined by endoscope 16 such that the pressurized fluid from AER 10 can fluid into the individual channels of the endoscope via the flexible conduits. A variety of different connector configurations can be used to make a mechanical and/or fluid connection between AER 10 (e.g., a fluidly conduit in fluid communication with the machine) and the individual channels and/or ports of endoscope 16, such as threaded connectors, bayonet connectors, cam and groove connectors, and the like. In either case, each of the one or more connections made by the operator between AER 10 and the individual channels and/or ports of endoscope 16 is desirably fluid tight to prevent fluid intended to be passed through the corresponding channel of the endoscope from bypassing at the connection location.

In operation, an operator may perform one or more manual reprocessing steps on endoscope 16 prior to introducing the endoscope in AER 10. The type of manual cleaning activities may be performed on the endoscope include disassembly and removal of components, applying brushes to clear channels, wiping to remove visible liquids and solids, and other human-performed cleaning actions. After completion of any desired manual reprocessing steps, the operator can introduce endoscope 16 into processing chamber 14 of AER 10 for automated reprocessing. In some examples, the operator may insert endoscope 16 into an endoscope carrier and fluidly connect one or more (e.g., optionally all) of the individual channels of the endoscope to a multi-port connector carried by the carrier. For example, the operator may fluidly connect the inlet of a biopsy channel of endoscope 16 and the inlet of a suction channel of the endoscope to the multi-port connector carried by the carrier. The operator can then insert the carrier into processing chamber 14, with the multi- port connector mechanically and/or fluidly connecting to a corresponding connector within processing chamber 14. Additionally or alternatively, the operator can insert endoscope 16 into processing chamber 16 and connect one or more (e.g., optionally all) of the individual channels of the endoscope, including the inlet of a biopsy channel and the inlet of a suction channel, to corresponding connection lines of AER 10 to place the channels in fluid communication with the machine.

AER 10 can include a user interface 24 that an operator can interact with to input information for controlling the AER and/or to output information back to the operator. User interface 24 may be implemented using a presence-sensitive display, such as a resistive touchscreen, a surface acoustic wave touchscreen, a capacitive touchscreen, a projective capacitance touchscreen, a pressure sensitive screen, an acoustic pulse recognition touchscreen, or another presence-sensitive display technology. User interface 24 may function as an output (e.g., display) device using any one or more display devices, such as a liquid crystal display (LCD), dot matrix display, light emitting diode (LED) display, organic light-emitting diode (OLED) display, or similar display capable of outputting visible information to the user. User interface 24 may include physically-depressible buttons that may receive tactile input from an operator using AER 10.

AER 10 can include one or more controller 26 that manage the overall operation of the AER. Controller 26 can be communicatively coupled to sensors, supply control devices (e.g., pumps, valves), and/or other controllable components of AER 10 to manage the overall operation of the machine. Controller 26 includes a processor and a memory. The memory can store software for running the controller and may also store data generated or received by the processor. The processor can run software stored in the memory to manage the operation of the device.

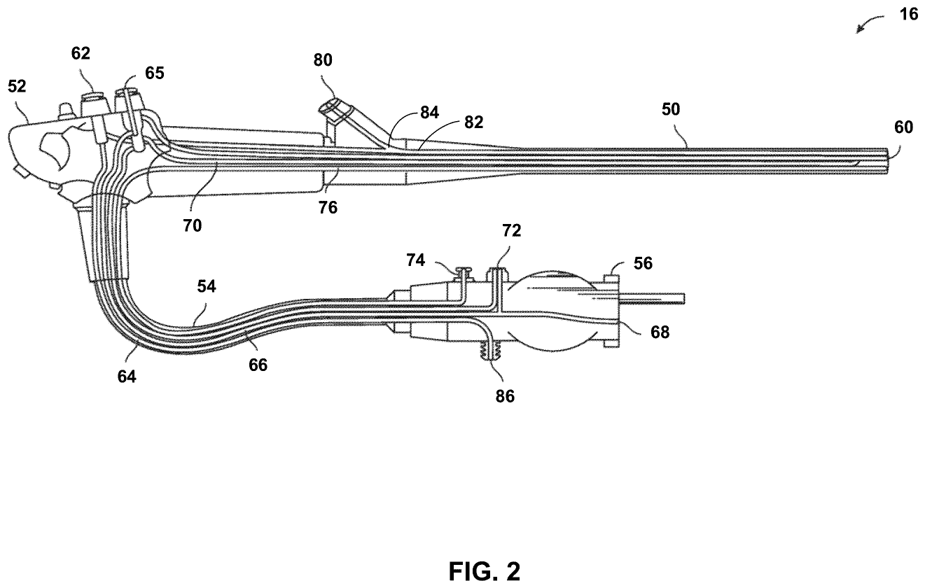

FIG. 2 is a schematic cross-section illustration of an example configuration of endoscope 16 that can be reprocessed according to example devices and techniques of the disclosure. Endoscope 16 as depicted includes portions that are generally divided into an insertion tube 50, a control section 52, a universal or umbilical cord 54, and a connector section 56 (which is sometimes referred to as a light guide section). Insertion tube 50 can extend from a proximal end to a distal end 60 and be configured for insertion into the body of a patient. A number of imaging, light, and stiffness components and related wires and controls used in endoscopes are not depicted for simplicity. Rather, FIG. 2 is intended to provide a simplified illustration of example endoscope channels. It will be understood that the presently discussed concepts can be applicable to other form factors and designs of endoscopes.

In the example of FIG. 2, the control section 52 hosts a number of controls used to actuate the positioning, shape, and behavior of endoscope 16. For instance, if the insertion tube 50 is flexible, the control section 52 may enable the operator to flex the insertion tube 50 based on patient anatomy and the endoscopic procedure. The control section 52 can include a suction valve 62 allowing the operator to controllably apply suction at the distal end 60 via a suction channel 64. The control section 52 can also include an air/water valve 65 which allows the distribution of air and/or water from an air channel 66 (provided from an air pipe source 68) or a water channel 70 (provided from a water source connected to a water source connector 72) to the distal end 60 of the insertion tube. The depicted design of endoscope 16 also includes a water jet connector 74 via a water-jet channel 76, to provide additional distribution of water separate from the air channel 66.

The universal cord 54 (also known as an “umbilical cable”) connects the connector section 56 to the control section 52 of the endoscope. The connector section 56 can provide a source of light which is distributed to the end of the insertion tube 50 using a fiber optic cable or other light guides. An imaging element (e.g. camera) used for capturing imaging data may be located at or in the connector section 56 or adjacent to the distal end 60 of the insertion tube.

Endoscope 16 in FIG. 2 is also illustrated as having a biopsy port 80 (e.g., biopsy valve) through which objects and/or instruments can be inserted into and guide down to and out of the distal end 60 of insertion tube 50. Biopsy port 80 is connected to suction channel 64. The portion of the suction channel 64 which extends from the biopsy port 80 to the distal end 60 of the insertion tube 50 is also known as the biopsy channel 82. Accordingly, the biopsy channel 82 extends from biopsy port 80 through a biopsy channel outlet at the distal end 60 of the insertion tube 50. The biopsy channel 82 branches between biopsy port 80 and a remained of suction channel 64 at a branch 84. The remainder of suction channel 64 extends proximally from the biopsy channel 82 through control section 52 and universal cord 54 back to a suction connector 86 located at the connector section 56. During use, a vacuum source can be applied to suction connector 86 to suck fluid and/or material into the distal end 60 of insertion tube 50 via the applied suction and control of suction valve 62.

During reprocessing of endoscope 16, an operator can connect biopsy port 80 to AER 10 (FIG. 1) to deliver one or more fluids via the connected biopsy port through biopsy channel 82 and out the distal end 60 of insertion tube 50. The connection between AER 10 and biopsy port 80 may be evaluated before, during, and/or after delivering one or more cleaning and/or disinfecting fluids through fluid line of the AER intended to be connected to the biopsy port to determine if the biopsy port is appropriately connected to the AER. For example, the AER may be connected to the biopsy port 80 of endoscope 16 and one or more test fluids introduced through the biopsy port into the endoscope. The one or more test fluids (which may be the same as or different than the actual fluids delivered to the channel during cleaning and/or disinfection) may flow through the insertion tube 50 and discharge out the distal end 60 of the insertion tube. In addition, because of the interconnection between biopsy channel 82 and suction channel 64, the one or more test fluids may also flow toward suction connector 86 via the suction channel (e.g., with suction valve 62 removed or held open to prevent the valve from blocking backflow from the biopsy port to the suction connector).

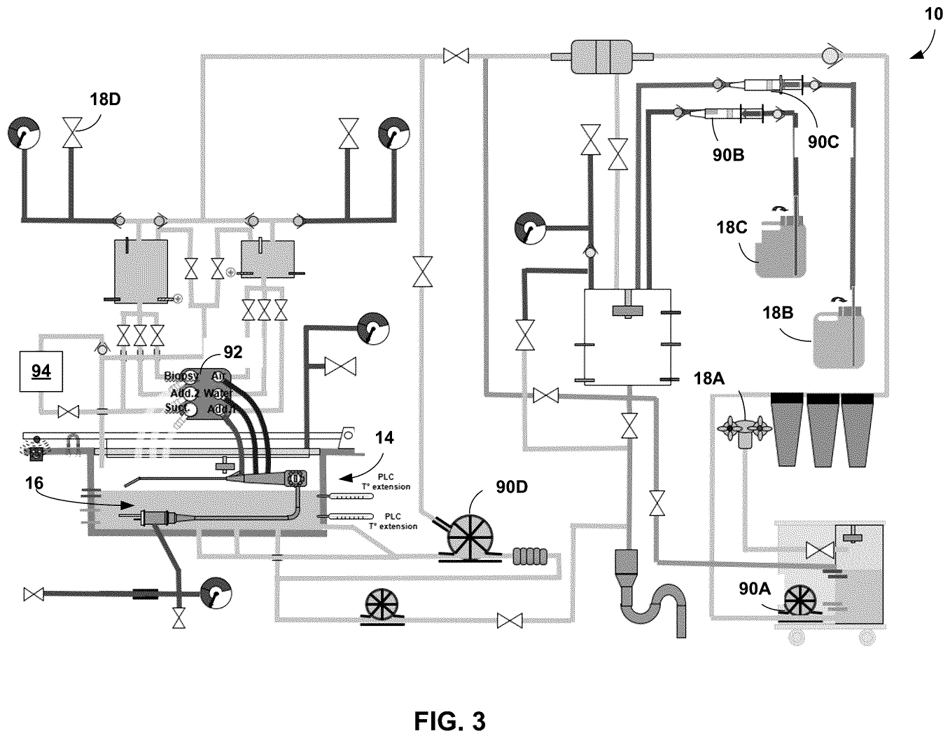

FIG. 3 is a schematic diagram showing an example configuration of AER 10 that can be used to detect connectivity of the biopsy channel 82 of endoscope 16 during an endoscope reprocessing procedure. As shown in this diagram, AER 10 includes previously described processing chamber 14 containing endoscope 16 for reprocessing. AER includes one or more test and/or reprocessing fluids 18 that can be delivered to the interior channels of endoscope 16 and/or exterior surface of the endoscope within processing chamber 14. In particular, in the illustrated example, AER 10 includes water 18A, first and second cleaning and/or disinfecting liquids 18B, 18C, and air 18D. The fluids 18 can be placed in selective fluid communication with one or more (e.g., optionally all) of the channels of endoscope 16 connected within processing chamber 14. One or more pumps 90A-90D can control fluid delivery within AER 10.

In the illustrated example, endoscope 16 is connected to AER 10 in processing chamber 14 via one or more connectors 92. As illustrated, connector 92 is illustrated as manifold having multiple ports to fluidly connect to the different channels of the endoscope. Fluid 18 can be delivered into the one or more connected channels of endoscope 16 via connector 92 (e.g., as AER 10 operates under the control of controller 26 discussed with respect to FIG. 1).

AER 10 in FIG. 3 is also illustrated as having at least one fluid detection sensor 94. Fluid detection sensor 94 can be fluidly connected to at least suction connector 86 of endoscope 16 via one or more connectors, flexible and/or inflexible fluid lines, and/or other components that communicate between the suction connector and the fluid detection sensor. Fluid detection sensor 94 can be communicatively coupled to controller 26. Fluid detection sensor 94 can be implemented using a variety of different types of sensors, including a flow sensor (e.g., flow switch), a pressure sensor, a capacitive sensor, and combinations thereof. Fluid detection sensor 94 can detect the presence of fluid at suction connector 86 and/or the amount of fluid flowing out of the suction connector and communicate information concerning the detected fluid (e.g., presence, amount) to controller 26.

During operation, an operator can load endoscope 16 into processing chamber 14 and fluidly connect one or more channels of the endoscope to AER 10. For example, the operator may fluidly connect biopsy port 80 of endoscope 16 with a fluid line of AER 10 configured to deliver cleaning and/or sanitizing fluids to biopsy channel 82. The operator may also fluidly connect suction connector 86 of endoscope 16 with fluid detection sensor 94. The fluid connection between suction connector 86 and AER 10 may or may not also be connected to one or more fluids 18 for delivering cleaning and/or sanitizing fluid(s) through the suction connector. While the connection between AER 10 and biopsy port 80 should be fluid tight, equipment or operator issues may result in the connection not being fluid tight (e.g., such that some or all of the fluid intended to be delivered into the biopsy port 80 via the connection instead discharges or leaks at the connection and does enter the biopsy port).

To evaluate the adequacy of the connection between the biopsy port 80 and AER 10, the AER may monitor signals form fluid detection sensor 94 fluidly connected to the biopsy port via suction channel 64. For example, before, during, and/or after initiating reprocessing of endoscope 16 in which one or more fluids 18 are delivered to the interior channels and/or exterior surface of the endoscope, AER may deliver fluid to biopsy port 80 and monitor for corresponding detection of the fluid at suction connector 86.

For example, operating under the control of controller 26, AER 10 may control one or more pumps 90, valves, and/or other fluid delivery features of the AER to introduce fluid into the fluid line and associated connection port of the machine intended to be connected to biopsy port 80. Example fluids that may be introduced into biopsy channel 82 include, but are not limited to, air and water.

To effectively test the connection between AER 10 and biopsy channel 82, the fluid may be delivered as suitable pressure and volume. In some examples, the fluid delivered to biopsy port 80 may be pressurized to a pressure of at least 1.5 bar, such as at least 2.0 bar, at least 2.5 bar, at least 3.0 bar, at least 4 bar, or at least 5bar. For example, the fluid may be pressurized to a pressure within a range from 2 bar to 5 bar. The rate at which the fluid is delivered may vary depending on the whether the fluid is a liquid (e.g., water) or a gas (e.g., air). For a comparatively large channel such as the biopsy channel, a liquid fluid may be delivered at a rate within a range from 0.1 L/min to 10 L/min, such as from 0.2 L/min to 5 L/min. A gas fluid may be delivered at a rate within a range from 5 L/min to 25 L/min, such as from 10 L/min to 20 L/min, or approximately 15 L/min.

In response to introducing fluid into biopsy port 80, the one or more fluid detection sensors 94 can detect the presence of fluid and/or the amount of fluid (e.g., flow rate, volume) in suction port 86. Controller 26 can receive data from the sensor 94 indicative of the detected fluid and/or amount of fluid. In some examples, controller 26 can analyze data indicative of the fluid measured by fluid detection sensor 94 and compared the data to information stored in memory. For example, controller 26 may analyze the flow rate and/or volume of fluid measured at suction port 86 and compare the measured flow rate and/or volume to one or more corresponding values associated with an adequate connect between biopsy port 80 and AER 10. In other examples, controller 26 may determine if fluid detection sensor 94 detected fluid in response to introducing fluid through the line intended to be connected to biopsy port 80 (in which case controller 26 determines that an adequate connection is established) or determine that the fluid detection sensor did not detect fluid in response to introducing fluid through the line intended to be connected to the biopsy port (in which case the controller determines that an adequate connection is not established).

If controller 26 determines that the connection between biopsy port 80 and AER 10 is sufficiently fluid tight based on the signal from fluid detection sensor 94 (e.g., by detecting fluid in suction port 86 and/or detecting a volume/flow rate of fluid corresponding to a fluid tight connection), controller 26 may control AER 10 to start or continue with reprocessing processes. For example, controller 26 may control the one or more pumps 90, valves, and/or other fluid delivery features of AER 10 to introduce fluid into internal channels of endoscope 16 and/or on the external surface of the endoscope to clean and/or disinfect the endoscope. If controller 26 determines that the connection between biopsy port 80 and AER 10 is not sufficiently fluid tight based on the signal from fluid detection sensor 94 (e.g., by failing to detect fluid in suction port 86 and/or detecting a volume/flow rate of fluid less than that corresponding to a fluid tight connection), controller 26 may take different control actions. For example, controller 26 may stop or prohibit AER 10 from proceeding with reprocessing of endoscope 16. Additionally or alternatively, controller 26 may control issuance of a user alert (e.g., via user interface 24) indicating that a fluid tight connection between the AER and biopsy channel was not detected.

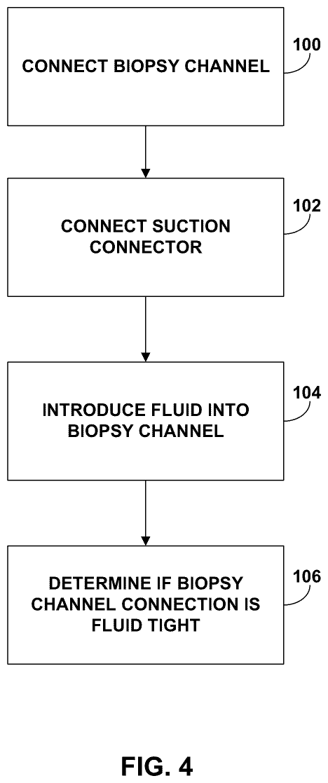

FIG. 4 is a flow diagram illustrating an example technique for detecting connectivity of a biopsy channel of an endoscope during an endoscope reprocessing procedure. At step 100 in FIG. 4, the example technique involves connecting a fluid supply line 22 of AER 10 to biopsy channel 82 of endoscope 16. For example, the operator can fluidly connect fluid supply line 22 of AER 10 to biopsy port 80 of endoscope 16. In some examples, the user inserts endoscope 16 into processing chamber 14 and thereafter connects the biopsy channel to the AER. In some examples, the operator fluidly connects biopsy port 80 of endoscope 16 to a multi-port connector carried by an endoscope carrier on which the endoscope is placed. The endoscope carrier is then inserted into processing chamber 14 and a connection established between the multi-port connector carried by the endoscope carrier and AER 10.

Step 102 in the example technique of FIG. 4 involves connecting fluid detection sensor 94 of AER 10 to suction connector 86 of endoscope 16. For example, the operator can fluidly connect a fluid line that is connected to fluid detection sensor 94 to suction connector 86 of endoscope 16. In some examples, the user inserts endoscope 16 into processing chamber 14 and thereafter connects the suction connector to the fluid detection sensor. In some examples, the operator fluidly connects suction connector 86 of endoscope 16 to a multi-port connector carried by an endoscope carrier on which the endoscope is placed. The endoscope carrier is then inserted into processing chamber 14 and a connection established between the multi-port connector carried by the endoscope carrier and AER 10 (e.g., the fluid detection sensor 94 of the AER).

With biopsy channel 82 fluidly connected to one or mor fluids 18 and suction connector 86 fluidly connected to fluid detection sensor 94 of AER 10, step 104 of the example technique of FIG. 4 involves introducing a fluid into fluid supply line 22 connected to biopsy channel 82 of endoscope 16. Controller 26 of AER 10 may control the one or more pumps 90, valves, and/or other fluid delivery features of the AER to deliver pressurized fluid 18 to fluid supply line 22, such as air and/or water (e.g., first testing with one fluid and then testing with the second fluid).

At step 106, controller 26 of AER 10 can determining if fluid supply line 22 is adequately connected to biopsy channel 82 of endoscope 16 based on detection of the fluid at suction connector 86 by fluid detection sensor 94. For example, the technique may involve either (a) detecting, by fluid detection sensor 94, the fluid introduced into fluid supply line 22 connected to biopsy channel 82 via discharge of the fluid from suction connector 86 or (b) failing to detect, by fluid detection sensor 94, the fluid introduced into fluid supply line 22 connected to biopsy channel 82 via discharge from suction connector 86. In some examples, fluid detection sensor 94 measures a volume and/or flow rate of fluid discharged from suction connector 86 and compares the measured volume and/or flow rate to threshold information stored in memory corresponding to an adequate fluid tight connection.

In either case, if AER 10 determines that the fluid connection between the machine and biopsy channel 82 of endoscope 16 is sufficiently fluid tight, the AER may start or continue with cleaning, sterilizing, and/or disinfecting steps on the endoscope. If AER 10 determines that the fluid connection between the machine and biopsy channel 82 of endoscope 16 is not sufficiently fluid tight, the AER may stop or prohibit starting with cleaning, sterilizing, and/or disinfecting steps on the endoscope and/or issue one or more user alerts informing the operator of the connection issue.

The techniques described in this disclosure may be implemented, at least in part, in hardware, software, firmware or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in a computer-readable medium, such as a non-transitory computer-readable storage medium, containing instructions. Instructions embedded or encoded in a computer-readable storage medium may cause a programmable processor, or other processor, to perform the method, e.g., when the instructions are executed. Non-transitory computer readable storage media may include volatile and/or non-volatile memory forms including, e.g., random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

1. A method of detecting connectivity of a biopsy channel of an endoscope during an endoscope reprocessing procedure, the method comprising:
connecting a fluid supply line of an endoscope reprocessing machine to a biopsy channel of an endoscope;
connecting a fluid detection sensor of the endoscope reprocessing machine to a suction connector of the endoscope;
introducing a fluid into the fluid supply line connected to the biopsy channel of the endoscope; and
determining if the fluid supply line is adequately connected to the biopsy channel of the endoscope based on detection of the fluid by the fluid detection sensor.

2. The method of claim 1, wherein determining if the fluid supply line is adequately connected to the biopsy channel of the endoscope based on detection of the fluid by the fluid detection sensor comprises either (a) detecting, by the fluid detection sensor, the fluid introduced into the fluid supply line connected to the biopsy channel via discharge from the suction connector of the endoscope or (b) failing to detect, by the fluid detection sensor, the fluid introduced into the fluid supply line connected to the biopsy channel via discharge from the suction connector of the endoscope.

3. The method of claim 1, further comprising, in response to detecting the fluid by the fluid detection sensor, the endoscope reprocessing machine proceeding to perform a cleaning procedure on the endoscope.

4. The method of claim 1, further comprising, in response to determining that the fluid supply line is not adequately connected to the biopsy channel, the endoscope reprocessing machine one or both of issuing a user alert and prohibiting proceeding with a cleaning procedure on the endoscope.

5. The method of claim 1, wherein:
the endoscope comprises an insertion tube extending from a proximal end to a distal end configured to be inserted into a body of a patient, a control section, a connector section, and an umbilical cord extending between the control section and the connector section;
the biopsy channel extends from a biopsy port at the control section through a biopsy channel outlet at a distal end of the insertion tube; and
the suction connector is located at the connector section.

6. The method of claim 5, wherein connecting the fluid supply line of the endoscope reprocessing machine to the biopsy channel of the endoscope comprises connecting the fluid supply line to the biopsy port.

7. The method of claim 1, wherein connecting the fluid detection sensor to the suction connector of the endoscope comprises connecting a fluid receiving line of the endoscope reprocessing machine to the suction connector of the endoscope, the fluid receiving line being in fluid communication with the fluid detection sensor.

8. The method of claim 1, wherein the fluid is water.

9. The method of claim 1, wherein the fluid is air.

10. The method of claim 1, wherein the fluid is pressurized to at least 2 bar and delivered at a rate of at least 5 L/min.

11. The method of claim 1, wherein the fluid detection sensor is one of a fluid contact sensor, a flow sensor, and a pressure sensor.

12. The method of claim 1, wherein:
the endoscope reprocessing machine comprises a processing chamber configured to receive the endoscope and deliver a cleaning chemistry to one or both of an internal lumen and an external surface of the endoscope;
connecting the fluid supply line of the endoscope reprocessing machine to the biopsy channel of the endoscope comprises connecting the fluid supply line with the endoscope located in the processing chamber; and
connecting the fluid detection sensor of the endoscope reprocessing machine to the suction connector of the endoscope comprise connecting the fluid detection sensor with the endoscope located in the processing chamber.

13. An endoscope reprocessing machine comprising:
a processing chamber configured to receive an endoscope to be reprocessed;
a fluid supply line configured to connect to a biopsy channel of the endoscope;
a fluid detection sensor configured to connect to a suction connector of the endoscope; and
a controller configured to:
control introduction of a fluid into the fluid supply line connected to the biopsy channel of the endoscope; and
determine if the fluid supply line is adequately connected to the biopsy channel of the endoscope based on detection of the fluid by the fluid detection sensor.

14. The machine of claim 13, wherein the controller is configured to determine if the fluid supply line is adequately connected to the biopsy channel of the endoscope based on detection of the fluid by the fluid detection sensor comprises by at least either (a) detecting, by the fluid detection sensor, the fluid introduced into the fluid supply line connected to the biopsy channel via discharge from the suction connector of the endoscope or (b) failing to detect, by the fluid detection sensor, the fluid introduced into the fluid supply line connected to the biopsy channel via discharge from the suction connector of the endoscope.

15. The machine of claim 13, wherein the controller is configured, in response to detecting the fluid by the fluid detection sensor, to control the endoscope reprocessing machine to proceed to perform a cleaning procedure on the endoscope in the processing chamber.

16. The machine of claim 13, wherein the controller is configured, in response to determining that the fluid supply line is not adequately connected to the biopsy channel, to control the endoscope reprocessing machine to one or both of issue a user alert and prohibit proceeding with a cleaning procedure on the endoscope in the processing chamber.

17. The machine of claim 13, wherein the fluid supply line is configured to connect to the biopsy channel of the endoscope by connecting to a biopsy port on the endoscope.

18. The machine of claim 13, wherein the fluid is one or both of air and water.

19. The machine of claim 13, wherein the fluid detection sensor is one of a fluid contact sensor, a flow sensor, and a pressure sensor.

20. The machine of claim 13, wherein the controller is configured to control introduction of the fluid into the fluid supply line that is pressurized to at least 2 bar and delivered at a rate of at least 10 L/min.
